(19) United States
(12) Patent Application Publication
(10) Pub. No.: US 2005/0074895 A1
(43) Pub. Date: Apr. 7, 2005

(54) Alkyl-substituted vinyl pyridines for mass-coded tagging of proteins

(75) Inventor: Lee Olech, Rodeo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BIO-RAD LABORATORIES, INC., Hercules, CA (US)

(21) Appl. No.: 10/931,943

(22) Filed: Aug. 31, 2004

Related U.S. Application Data
(60) Provisional application No. 60/500,596, filed on Sep. 5, 2003.

(51) Int. Cl. G01N 33/00; C07K 14/47

(52) U.S. Cl. 436/93; 530/409

(57) ABSTRACT

Comparative studies of proteins from two or more cell populations are performed by alkylating the cysteine thiol groups of the proteins of all populations with vinyl pyridines in which the pyridine ring in at least one of the populations, in addition to the vinyl substituent, bears one or more alkyl substituents, the various populations thus differing in the alkyl substituents on the pyridine ring. Proteins of one population are thus alkylated with vinyl pyridine itself and those of another population with an alkyl-substituted vinyl pyridine, or both with alkyl-substituted vinyl pyridines using different alkyl groups or a different number of alkyl groups. The mass differential afforded by the difference in alkyl substitution affords a means of pairing individual proteins of one population with those of another while differentiating between the two, using analytical techniques disclosed in the prior art for isotope-coded affinity tagging.

### CROSS REFERENCE TO RELATED APPLICATION

This application claims benefit from U.S. Provisional Patent Application No. 60/500,596, filed Sep. 5, 2003, the contents of which are incorporated herein by reference in their entirety.

### BACKGROUND OF THE INVENTION

1. Field of the Invention

This invention resides in the chemistry of proteomics, and in particular, the differential determinations of protein expression between cells.

2. Description of the Prior Art

The quantitative analysis of protein expression is a means for studying variations between biological cells. These variations can provide valuable information toward determining the differences between cells as well as the ways in which cells respond to different environments, to biological processes, and to treatments with agents such as other cells and biological organisms, viruses, drugs, enzymes, oligopeptides and polypeptides in general, and generally anything with the potential to influence or change the normal biological functioning of a cell, for the better or the worse, either intentionally or unintentionally such as, for example, the result of a disease, genetic variation, or other abnormality.

The comparative analysis of protein expression between different cells is an extension of the field of proteomics, whose origin lies in studies designed to map out the complete protein complement of particular organelles, cells, or tissues. Although comparative analysis is still in its infancy, one method of performing the analysis is isotope-coded affinity tagging (ICAT), a procedure in which proteins from one cell population are tagged with an isotope which provides those proteins with masses that differ by a uniform differential from the masses of the untagged proteins of the other cell population. The tagged and untagged cell populations, representing the two cell populations to be compared with each other, are then combined, typically in equal amounts, and the combined population is analyzed by any method that can reveal the ratio of any two molecular components that differ only by the differential that is attributable to the presence of the isotope. Methods of analysis include two-dimensional gel electrophoresis in combination with tandem mass spectrometry, and microcapillary liquid chromatography in combination with data-dependent electrospray ionization tandem mass spectrometry. Descriptions of ICAT and of the use of these analytical techniques in association with ICAT appear in U.S. Patent Application Publication No. US 2002/0076739 A1, publication date Jun. 20, 2002 (Aebersold, R. H., et al., assigned to University of Washington and entitled “Rapid Quantitative Analysis of Proteins or Protein Function in Complex Mixtures”) and by Patton, W. F., et al., “Two-dimensional gel electrophoresis; better than a poke in the ICAT?,” Current Opinion in Biotechnology 13:321-328 (2002), and references cited therein. The contents of both published documents cited in this paragraph and the references cited in them, as well as those cited in other parts of this specification, are incorporated herein by reference in their entirety for all legal purposes capable of being served thereby.

The isotopic tag in these procedures is described in the prior art as any stable isotope of an atom which when substituted for the common form of the atom results in a tagged protein that is substantially chemically identical and yet isotopically distinguishable from the untagged protein. The most common and convenient means of isotope tagging is the substitution of deuterium for one or more of the hydrogen atoms in the protein or in a derivative of the protein if the protein is derivatized during the procedure. Since the procedures of the prior art typically involve derivatization of the proteins for any of various reasons, isotopic tagging is achieved by using a tagged derivatizing agent for one cell population and an untagged derivatizing agent for the other cell population. In the Aebersold et al. disclosure, for example, the proteins are derivatized by attachment of an affinity label through a linker group, the affinity label being used for isolation of the proteins, or of a representative subgroup of the proteins, from the cell materials as a whole and thereby facilitating the analysis of individual proteins. The isotope is typically a part of the linker group, and in cases where the affinity label is removed prior to analysis, the removal is achieved by cleavage of the linker or the affinity label in a manner whereby the isotope remains in the portion of the linker that remains bound to the protein. Linker groups disclosed in the prior art include ethers, polyethers, ether diamines, polyether diamines, diamines, amides, polyamides, polythioethers, disfulfides, silyl ethers, alkyl chains, alkenyl chains, aryl groups, diaryl groups, and alkyl-aryl groups. In the Patton et al. disclosure referenced above, and in references cited therein, the proteins are derivatized by attachment of a label through a group that is specific for cysteine thiol residues. When the derivatized proteins are then detected and analyzed to the exclusion of those that are not derivatized, the analysis is directed to a much reduced number of proteins, thereby simplifying the analysis while still obtaining results that are representative of the protein complement as a whole. Derivatization of the cysteine thiol residues also eliminates the appearance of spurious protein zones in electrophoresis that are the result of disulfur bridges between proteins. When cysteine thiol derivatization is performed, the isotope is typically included in the derivatization agent.

Regardless of how the isotope is associated with the proteins, the number of isotopes associated with each protein is a matter of choice, the greater the number of isotopes the larger the mass difference and the easier and more reliable the differential analysis. Despite this versatility, isotopic tagging suffers from the difficulties and expense of obtaining isotopic reagents and the limitations on the types of reagents that can be synthesized with an isotope. Also, the mass difference obtained by isotopic tagging is small, even with multiple isotopes. When eight hydrogen atoms are replaced by deuterium, for example, as in much of the prior art, the mass difference is only that of eight neutrons. This limits the choice of analytical methods that can identify the proteins while differentiating between the tagged and untagged proteins by the increased mass of the tag.

### SUMMARY OF THE INVENTION

This invention resides in the mass-coded tagging of proteins for comparative analysis of protein expression by the reaction of proteins with alkyl-substituted vinyl pyridines as thiol alkylating agents. Proteins that have been alkylated with these agents are compared with proteins that have been alkylated with vinyl pyridine itself or other alkyl-substituted vinyl pyridines of different molecular weight in any of the comparative analytical procedures referenced above, the weight difference attributable to the difference in alkyl groups, or to the presence of the alkyl group in one case and its absence in the other, afford the distinguishable difference on which the comparative analysis relies. The mass difference is considerably greater than that achieved by isotope tagging.

This invention resides in processes for the comparative analysis of different protein populations by coupling the proteins to the alkyl-substituted vinyl pyridines and analyzing the populations after coupling while differentiating between them by mass difference. This invention also resides in kits that contain a plurality of the alkyl-substituted vinyl pyridines for coupling to different populations of proteins.

### BRIEF DESCRIPTION OF THE FIGURE

The attached FIGURE is a plot of DTNB optical density vs. time for 4-methyl-2-vinyl pyridine and 2-vinylpyridine, as a comparative demonstration of alkylation kinetics for the two species.

### DETAILED DESCRIPTION OF THE INVENTION AND PREFERRED EMBODIMENTS

The alkyl-substituted vinyl pyridines used in the practice of this invention are represented by the formula 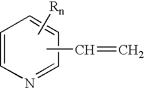
in which R is lower alkyl, and n is 1 to 4. The term “lower alkyl” in this context is intended to mean saturated alkyl groups of six carbon atoms or less, preferably four carbon atoms or less. Particularly preferred alkyl groups are those of two carbon atoms or less, i.e., methyl and ethyl. For alkyl groups of three or more carbon atoms, the term includes both straight-chain and branched-chain alkyl groups, preferably branched-chain such as isopropyl, isobutyl and tertiary butyl. Preferred values of n are 1 and 2. As indicated by the formula, the vinyl group and the alkyl group(s) may be bonded to any of the five available carbon atoms of the pyridine ring. The vinyl group can thus be bonded to the 2-carbon, the 3-carbon, or the 4-carbon. In preferred embodiments, however, the vinyl group is bonded to either the 2-carbon or 4-carbon, i.e., to the carbon adjacent to or opposite the N atom. Likewise in preferred embodiments, the alkyl group(s) R is bonded to the 2-, 4-, or 6-carbon of the ring, as available, i.e., those that are not bonded to the vinyl group. Specific examples of thiol alkylating agents within this formula are 2-vinyl-4-methylpyridine, 2-vinyl-6-methyl pyridine, 4-vinyl-2-methylpyridine, 2-vinyl-4,6-methylpyridine, and 4-vinyl-2,6-methyl pyridine.

These alkylating agents can be prepared by methods in the literature, such as for example, those described by Bodalski, R., et al., “Alkyl- and Alkenyl-Pyridines. Part XI. Studies on Synthesis of Divinylpyridines. Reactions of 2,4-Lutidine and 2,4,6-Collidine With Formaldehyde,” Roczniki Chemii Ann. Soc. Chim. Polonorum 40, 1505-1517 (1966), and references cited therein. A preferred method is one involving a reaction between formaldehyde (or paraformaldehyde) with a methyl alkylpyridine in which the methyl group occupies the position to be occupied by the vinyl group in the desired product, to yield an intermediate in which the methyl group is replaced by a 2-hydroxyethyl group, followed by reaction of the intermediate with a strong base to convert the 2-hydroxyethyl goup to a vinyl group. Thus, the starting material for the synthesis of 2-vinyl-4-methylpyridine according to this procedure is 2,4-dimethylpyridine (i.e., 2,4-lutidine), and the starting material for the synthesis of 2-vinyl-4,6-methylpyridine is 2,4,6-collidine. The strong base can be potassium hydroxide. Both reactions are generally performed at elevated temperatures, using appropriate solvents and other reaction conditions, readily determinable by routine methods. Other synthesis methods will be apparent to those skilled in the art.

The use of the alkyl-substituted vinyl pyridines in the practice of this invention in the comparative analysis of protein expression is accompanied by the use of corresponding vinyl pyridines that lack the alkyl substituent, or that have a greater or lesser number of alkyl substituents, or that have alkyl substituents of a greater or lesser number of carbon atoms, or any combination of these variations that will produce a mass differential detectable by the analytical method of choice. Accordingly, the protein populations in general are coupled to vinyl pyridines of the above formula in which n is 0 to 4, each population having a fixed value of R and n throughout the population and the various populations differing by the choice of R, the value of n, or both. A preferred combination of coupling agents is that of an alkyl-substituted vinyl pyridine with the corresponding vinyl pyridine itself (not otherwise alkylated), the vinyl group occupying the same position in both agents. Vinyl pyridines are readily available from commercial sources, and their synthesis and use is described by Friedman, M., in “Application of the S-Pyridylation Reaction to the Elucidation of the Structures and Functions of Proteins,” J. Protein Chemistry 20(6):431-453 (2001) and references cited therein.

The tagging of the proteins with these agents is achieved by conventional methods disclosed in the prior art as referenced above. In general, the reaction is conducted under reducing conditions in an appropriate solvent, and ambient temperature will suffice, i.e., the reaction can be performed without external heating.

The following example is offered for purposes of illustration, and is not intended to impose any limits on the scope of the invention.

### EXAMPLE

The compound 2-vinyl-4-methylpyridine was prepared according to the method of Bodalski, R., et al., “Alkyl- and Alkenyl-Pyridines. Part XI. Studies on Synthesis of Divinylpyridines. Reactions of 2,4-Lutidine and 2,4,6-Collidine With Formaldehyde,” Roczniki Chemii Ann. Soc. Chim. Polonorum 40, 1505-1517 (1966). The procedure is shown in the following reaction scheme and described below.

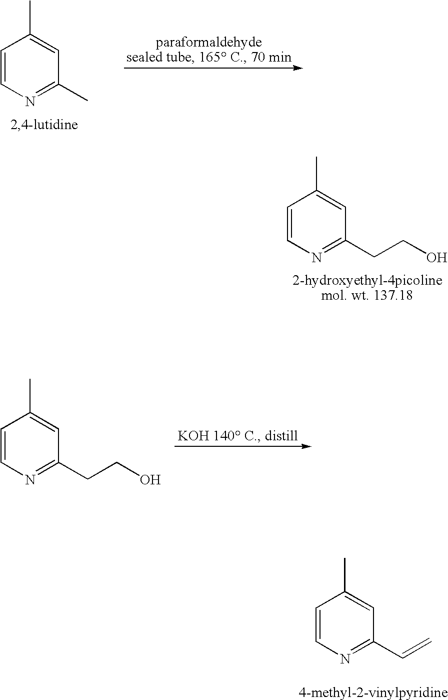

Four glass reaction vessels were charged with 2,4-lutidine (107 g, 1 mole), paraformaldehyde (30 g, 1 equivalent), hydroquinone (1.35 g), and water (1.65 mL), filling each vessel to about one-third capacity. The vessels were then sealed and their contents were heated to 165° C. and held at that temperature for 2 hours. (The theoretical yield of 4-methyl-2-(2′-hydroxyethyl)-pyridine was 137.2 g.) The reaction mixture was then distilled, starting at 33° C. and approximately 5 mm Hg, and a first fraction was collected up to 40° C. and 4 mm Hg, and the product distilled at 110° C. and 5 mm Hg. The product, a viscous oil, subsequently solidified to give 25.9 g of product (19% yield).

To this product, 5 g of solid KOH was added and the mixture heated to 140° C. for 15 minutes at atmospheric pressure, and then under vacuum (85 mm Hg). A small fore-run of water was collected, and the product was then distilled at 85-90° C. The product weighed 14.7 g (65% of theory). The resulting material, a mobile liquid, was stored at −20° C., stabilized with 100 ppm hydroquinone. Its structure was confirmed by NMR as that of 2-vinyl-4-methylpyridine.

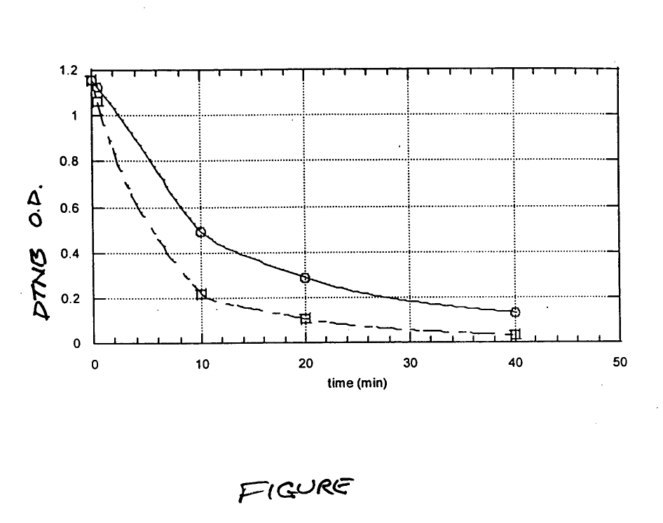

The alkylation kinetics of 2-vinyl-4-methylpyridine are compared with those of vinylpyridine in FIG. 1, where the dashed line (joining the square data points) represents 2-vinyl-4-methylpyridine and the solid line (joining the circular data points) represents vinylpyridine.

1. A process for the comparative analyses of each of a plurality of protein populations, said process comprising
(a) coupling proteins from each population at cysteine thiol groups to an alkyl-substituted vinyl pyridine of the formula 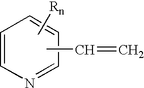
in which R is lower alkyl and n is 0 to 4, R and n are uniform for each population of said plurality, and at least one of R and n differs between populations of said plurality, such that the proteins of any one population upon being so coupled are distinguishable from the proteins of all other populations of said plurality by the mass difference caused by the difference in at least one of R and n; and
(b) analyzing said protein populations so coupled while differentiating between individual populations by said mass difference.

2. The process of claim 1 wherein n is 1 or 2.

3. The process of claim 1 wherein the —CH═CH₂ of said formula is bonded to the carbon atom at either the 2-position or the 4-position of the pyridine ring.

4. The process of claim 1 wherein the R group is bonded to the carbon atom at the 2-position, the 4-position, or the 6-position of the pyridine ring.

5. The process of claim 1 wherein the alkyl-substituted vinyl pyridine is a member selected from the group consisting of is a member selected from the group consisting of 2-vinyl-4-methylpyridine, 2-vinyl-6-methylpyridine, 4-vinyl-2-methylpyridine, 2-vinyl-4,6-methylpyridine, and 4-vinyl-2,6-methylpyridine.

6. A kit for the comparative analyses of each of a plurality of protein populations, said kit comprising a plurality of distinct species of alkyl-substituted vinyl pyridines of the formula 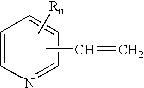
in which R is lower alkyl and n is 0 to 4, R and n are uniform for each species of said plurality, and at least one of R and n differs between species of said plurality, such that when coupled to a protein at a cysteine thiol group on said protein, the proteins coupled to any one species of said plurality are distinguishable from the proteins coupled to all other species of said plurality by the mass difference caused by the difference in at least one of R and n.

7. The kit of claim 6 wherein n is 1 or 2.

8. The kit of claim 6 wherein the —CH═CH₂ of said formula is bonded to the carbon atom at either the 2-position or the 4-position of the pyridine ring.

9. The kit of claim 6 wherein R group is bonded to the carbon atom at the 2-position, the 4-position, or the 6-position of the pyridine ring.

10. The kit of claim 6 wherein the alkyl-substituted vinyl pyridine is a member selected from the group consisting of is a member selected from the group consisting of 2-vinyl-4-methylpyridine, 2-vinyl-6-methylpyridine, 4-vinyl-2-methylpyridine, 2-vinyl-4,6-methylpyridine, and 4-vinyl-2,6-methylpyridine.
